(19) United States
(12) Patent Application Publication
(10) Pub. No.: US 2015/0005260 A1
(43) Pub. Date: Jan. 1, 2015

(54) METHODS FOR THE DETECTION AND THE TREATMENT OF DIASTOLIC DYSFUNCTION

(71) Applicants: INSERM (INSTITUT NATIONAL DE LA SANTE ET DE LA RECHERCHE MEDICALE), Paris (FR); UNIVERSITE DE LORRAINE, Nancy Cedex (FR); Centre Hospitalier et Universitaire de Nancy (CHU), Nancy Cedex (FR)

(72) Inventors: Faiez Zannad, Vandoeuvre Les Nancy (FR); Patrick Rossignol, Vandoeuvre Les Nancy (FR)

(21) Appl. No.: 14/370,142

(22) PCT Filed: Jan. 2, 2013
(86) PCT No.: PCT/EP2013/050008

§ 371 Date: Jul. 1, 2014

(30) Foreign Application Priority Data
Jan. 2, 2012 (EP) 12305003.1

(51) Int. Cl. G01N 33/68

(52) U.S. Cl. CPC G01N 33/6893; G01N 2800/325
USPC 514/91; 514/423; 514/212.07; 514/221; 514/530; 514/392; 514/412; 514/311; 514/409; 514/211.03; 435/7.92

(57) ABSTRACT

The present invention relates to methods and kits for detecting diastolic dysfunction in subjects without clinical signs of heart failure.

### FIELD OF THE INVENTION

The present invention relates to methods and kits for detecting diastolic dysfunction in subjects without clinical signs of heart failure.

### BACKGROUND OF THE INVENTION

More than 50% of heart failure (HF) patients present without a major deficit of left ventricular (LV) systolic function and are presumed to suffer from diastolic HF (DHF) because diastolic LV distensibility is usually impaired in these patients. DHF is also referred to as HF with normal left ventricular ejection fraction (HFNEF) or HF with preserved left ventricular ejection fraction (HFPEF). With an annual mortality rate of 22%, the prognosis of DHF is as grim as the prognosis of HF with a systolic function deficit (SHF), also labelled HF with reduced left ventricular ejection fraction (HFREF). In cross-sectional registries, a notable feature of DHF is its frequent association (˜80%) with overweight (BMI >25 kg/m²) or obesity (BMI >30 kg/m²). As overweight and obesity pose a major risk for arterial hypertension and type 2 diabetes mellitus (DM), DHF is also often associated with hypertension (˜75%) and DM (˜50%).

Among areas in need of major progress is ensuring accurate and early diagnosis of diastolic heart dysfunction in high risk patients (metabolic syndrome), before cardiac failure becomes clinically overt. Indeed, the accuracy and time of diagnosis by the usual clinical and echocardiography imaging approach is often inadequate. To study properly the epidemiology and prognosis and to optimize the prevention and treatment of heart failure, uncertainties and delays related to diagnosis must be minimized or avoided. A number of available biomakers have so far been used as tools for pathophysiological investigations and/or for risk stratification. However, these failed to be translated into important new individual diagnostic tools.

However, there is a need to develop new methods that will be suitable for detecting diastolic dysfunction in a subject without clinical sign of heart failure.

### SUMMARY OF THE INVENTION

The present invention relates to a method for detecting diastolic dysfunction in a subject without clinical signs of heart failure, comprising measuring PIIINP level in a blood sample obtained from said subject.

### DETAILED DESCRIPTION OF THE INVENTION

The inventors have investigated the association between blood biomarkers i;e concentrations of collagen synthesis and degradation and early alterations in cardiovascular structure and function in otherwise healthy abdominal obesity (AO) subjects, age and gender matched with healthy volunteers. The inventors surprisingly found that collagen peptide, PIIINP, a marker of collagen synthesis, is a positive explanatory factor of diastolic dysfunction in asymptomatic subjects without clinical signs of heart failure and without LVH (left ventricle hypertrophy) independently from the diastolic blood pressure (DBP). It therefore appears imperative to seek better management of said subjects, especially AO subjects, right from the earliest stages of the disease (i.e. subjects without clinical signs of heart failure).

### Prognostic and Diagnostic Methods According to the Invention:

The present invention relates to a method for detecting diastolic dysfunction in a subject without clinical signs of heart failure, comprising measuring PIIINP level in a blood sample obtained from said subject.

As used herein, the phrase, “diastolic dysfunction” has its general meaning in the art and refers to a condition in which abnormalities in mechanical function are present during diastole and which can occur in the presence or absence of heart failure and can co-exist with or without abnormalities in systolic function (Zile et al., JACC 41: 1519-1522 (2003)).

As used herein, the phrase “heart failure”, refers to any condition that can result from any structural or functional cardiac disorder that impairs the ability of the heart (e.g., the ventricle) to fill with or eject blood.

Typically a subject without clinical signs of heart failure is a subject classified at stage A of heart failure. The development of HF can be characterized by considering 4 stages of the disease. The first stage, Stage A, is a subject at high risk for HF but without structural heart disease or signs or symptoms of HF (for example, these are patients with hypertension, atherosclerotic disease, diabetes, obesity, metabolic syndrome or patients using cardiotoxins). The second stage, Stage B, is a subject having structural heart disease but without signs or symptoms of HF (for example, LV concentric remodeling as defined by an increased cardiac remodelling index represented by the ratio of LV mass/LV end-diastolic volume). The third stage, Stage C, is a subject having structural heart disease with prior or current symptoms and signs of HF (for example, these are patients who have known structural heart disease and exhibit shortness of breath and fatigue and have reduced exercise tolerance and display pulmonary rales and/or oedema). The fourth and final stage, Stage D, is refractory HF requiring specialized interventions (for example, patients who have marked symptoms at rest despite maximal medical therapy, namely, those who are recurrently hospitalized or cannot be safely discharged from the hospital without specialized interventions).

In a particular embodiment, the subject is an obese subject, and preferably a subject with abdominal obesity (>94 cm for male and >80 cm for female), and more preferably a subject with abdominal obesity without hypertension.

In one embodiment, the present invention relates to a method for detecting diastolic dysfunction in a subject without LVH (left ventricle hypertrophy), comprising measuring PIIINP level in a blood sample obtained from said subject.

As used herein, the phrase “left ventricle hypertrophy (LVH)” has its general meaning in the art and refers to any hypertrophy of the myocardium of a ventricle.

By “blood sample” is meant a volume of whole blood or fraction thereof, eg, serum, plasma, etc.

Measuring the PIIINP level may be assessed by any of a wide variety of well-known methods for measuring peptides.

In a particular embodiment, the methods of the invention comprise contacting the blood sample with a binding partner capable of selectively interacting with PIIINP present in the blood sample. The binding partner may be an antibody that may be polyclonal or monoclonal, preferably monoclonal. In another embodiment, the binding partner may be an aptamer.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985).

Alternatively, techniques described for the production of single chain antibodies (see e.g. U.S. Pat. No. 4,946,778) can be adapted to produce anti-PIIINP, single chain antibodies. Antibodies useful in practicing the present invention also include anti-PIIINP fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to PIIINP. For example, phage display of antibodies may be used. In such a method, single-chain Fv (scFv) or Fab fragments are expressed on the surface of a suitable bacteriophage, e. g., M13. Briefly, spleen cells of a suitable host, e. g., mouse, that has been immunized with a peptide are removed. The coding regions of the VL and VH chains are obtained from those cells that are producing the desired antibody against the peptide. These coding regions are then fused to a terminus of a phage sequence. Once the phage is inserted into a suitable carrier, e. g., bacteria, the phage displays the antibody fragment. Phage display of antibodies may also be provided by combinatorial methods known to those skilled in the art. Antibody fragments displayed by a phage may then be used as part of an immunoassay.

In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. 1997. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of conformationally constrained antibody variable regions displayed by a platform protein, such as E. coli Thioredoxin A, that are selected from combinatorial libraries by two hybrid methods (Colas et al., 1996).

The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited radioactive atom for scintigraphic studies such as I123, I124, In111, Re186, Re188.

The afore mentioned assays generally involve the binding of the binding partner (ie. antibody or aptamer) to a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

The level of biomarker peptide may be measured by using standard immunodiagnostic techniques, including immunoassays such as competition, direct reaction, or sandwich type assays. Such assays include, but are not limited to, agglutination tests; enzyme-labelled and mediated immunoassays, such as ELISAs; biotin/avidin type assays; radioimmunoassays; immunoelectrophoresis; immunoprecipitation.

More particularly, an ELISA method can be used, wherein the wells of a microtiter plate are coated with a set of antibodies which recognize said biomarker peptide. A blood sample containing or suspected of containing said biomarker peptide is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker peptide, the plate washed and the presence of the secondary binding molecule detected using methods well known in the art.

The method according to the invention may further comprise a step consisting of comparing the PIIINP level in the blood sample with a reference value, wherein detecting differential in the PIIINP level between the blood sample and the reference value is indicative that said subject has diastolic dysfunction.

In one embodiment, the reference value may be index value or may be derived from one or more risk prediction algorithms or computed indices for diastolic dysfunction. A reference value can be relative to a number or value derived from population studies, including without limitation, such subjects having similar body mass index, similar abdominal obesity, total cholesterol levels, LDL/HDL levels, systolic or diastolic blood pressure, subjects of the same or similar age range, subjects in the same or similar ethnic group. In one embodiment of the present invention, the reference value is derived from the level of PIIINP in a control sample derived from one or more subjects who are substantially healthy (i.e. having no diastolic dysfunction). In another embodiment, such subjects are monitored and/or periodically retested for a diagnostically relevant period of time (“longitudinal studies”) following such test to verify continued absence of diastolic dysfunction. Such period of time may be one year, two years, two to five years, five years, five to ten years, ten years, or ten or more years from the initial testing date for determination of the reference value. Furthermore, retrospective measurement of PIIINP levels in properly banked historical subject samples may be used in establishing these reference values, thus shortening the study time required, presuming the subjects have been appropriately followed during the intervening period through the intended horizon of the product claim. Typically, the levels of PIIINP in a subject who has diastolic dysfunction is deemed to be higher than the reference value obtained from healthy subjects who will develop diastolic dysfunction.

Typically, when the expression level of PIIINP measured according to the method of the invention is higher than the reference value, then it is concluding that the subject is afflicted or will be afflicted with diastolic dysfunction.

The method of the invention may also be particularly useful for monitoring the efficacy of a treatment for a diastolic dysfunction.

The method of the invention is particularly suitable for the management and the appreciation of risk for heart failure. Accordingly, the present invention relates to a method for determining whether a subject is at risk of developing diastolic heart failure comprising a step consisting of detecting diastolic dysfunction according to the method as above described.

### Therapeutic Methods and Uses:

Aldosterone receptor antagonists have demonstrated their impact on limiting extracellular matrix turnover. Serum level of PIIINP was significantly reduced after treatment with inhibitors of the renin angiotensin aldosterone system such as eplerenone and spironolactone (Zannad et al., 2000; Iraqi et al., 2009).

The present invention also relates to an inhibitor of the renin angiotensin aldosterone system for use in the prevention or treatment of diastolic dysfunction in a subject without clinical signs of heart failure having high level of PIIINP.

The term “inhibitor of the renin angiotensin aldosterone system” has it general meaning in the art. Such inhibitors may include but are not limited to renin inhibitors; Angiotensin converting enzyme (ACE) inhibitors; Angiotensin-II-receptor blocker;

Aldosterone synthase inhibitor; mineralocorticoid receptor antagonist; Dual angiotensin converting enzyme/neutral endopeptidase (ACE/NEP) inhibitor.

Examples of angiotensin converting enzyme (ACE) inhibitors include alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril.

Examples of Angiotensin-II-receptor blocker include valsartan, losartan, candesartan, telmisartan, eprosartan, olmesartan, irbesartan and tasosartan.

Examples of Aldosterone synthase inhibitor include non-steroidal aromatase inhibitors anastrozole, fadrozole (including the (+)-enantiomer thereof), as well as the steroidal aromatase inhibitor exemestane.

Examples of mineralocorticoid receptor antagonist include spironolactone and eplerenone.

Examples of Dual angiotensin converting enzyme/neutral endopeptidase (ACE/NEP) inhibitor include omapatrilate, fasidotril and fasidotrilate.

In one embodiment, the present invention relates to a method for preventing or treating diastolic dysfunction in a subject without clinical signs of heart failure in need thereof, comprising the steps of:

i) detecting diastolic dysfunction in said subject by the method according to the invention, and

ii) administering to said subject a therapeutically effective amount of an inhibitor of the renin angiotensin aldosterone system.

### Pharmaceutical Compositions:

Another object of the invention relates to a pharmaceutical composition comprising an inhibitor of the renin angiotensin aldosterone system and a pharmaceutically acceptable carrier for use in the prevention or treatment of diastolic dysfunction in a subject without clinical signs of heart failure having high level of PIIINP.

Typically, the inhibitor of the renin angiotensin aldosterone system may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitor of the renin angiotensin aldosterone system can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

Pharmaceutical compositions of the invention may include any further agent which is used in the prevention or treatment of diastolic dysfunction and heart failure. For example, the anti-diastolic dysfunction and anti-heart failure may include but are not limited to Angiotensin-converting enzyme (ACE) inhibitors, Angiotensin II receptor blockers, Beta blockers, Diuretics, Aldosterone antagonists, calcium channel blockers.

Pharmaceutical compositions of the invention may include any further agent which is used in the prevention or treatment of abdominal obesity.

In one embodiment, said additional active agents may be contained in the same composition or administrated separately.

In another embodiment, the pharmaceutical composition of the invention relates to combined preparation for simultaneous, separate or sequential use in the prevention or treatment of diastolic dysfunction.

### Kits:

A further object of the invention relates to kits for detecting diastolic dysfunction in a subject without clinical signs of heart failure, comprising means for measuring PIIINP level in a blood sample obtained from said subject.

In a particular embodiment, the kit of the invention further comprise means for comparing the PIIINP level in the blood sample with a reference value, wherein detecting differential in the PIIINP level between the blood sample and the reference value is indicative that said subject has diastolic dysfunction.

A further object of the invention relates to kits comprising an inhibitor of the renin angiotensin aldosterone system for use in the prevention or treatment of diastolic dysfunction in a subject without clinical signs of heart failure having high level of PIIINP.

A further object of the invention relates to kits comprising a pharmaceutical composition according to the invention and a pharmaceutically acceptable carrier for use in the prevention or treatment of diastolic dysfunction in a subject without signs of heart failure having high level of PIIINP.

The invention will be further illustrated by the following examples. However, these examples should not be interpreted in any way as limiting the scope of the present invention.

### EXAMPLE

Methods

Subjects' Selection

Between 40 and 65 years AO subjects (waist circumference >94 cm for male, >80 cm for female (Holt RIG, 2005) and age-gender matched healthy volunteers (body mass index <25 kg/m²) without AO were consecutively recruited through press advertisement. Known diabetic subjects or participants, with known or suspected hypertension at the screening visit (i.e. BP >140/90 mm Hg), morbid obesity (body mass index >40 kg/m²), personal history of cardiovascular events or of endocrine disease, inflammatory or neoplasic diseases were excluded. Written informed consent was obtained. Ethic committee (CPP Est-III) gave his consent to this study. No clinical trials.gov number was assigned to this study since it started in 2005.

Cardiac Phenotyping

Transthoracic doppler echocardiography (TTE) (HDI 5000) was performed and LV diastolic function was assessed with measurements of peak E wave, peak A wave, E/A ratio, deceleration time of E wave; doppler tissue imaging in the lateral part of mitral annulus: E′, A′ and E/E′ ratio. European society of echocardiography guidelines were used to classify DD (25). Diastolic dysfunction was diagnosed if E′ wave was less than 10 cm/s.

Cardiac MRI was performed on a 1.5-T magnet (Signa Excite, GE Medical Systems, Milwaukee, Wis., USA) equipped with an 8-element phased-array surface coil. A steady-state free precession pulse sequence was used to assess LV function in contiguous short axis planes, each slice being recorded during a ≦15-second breath-hold period (Codreanu et al., 2007; Mandry et al., 2009). Main acquisition parameters were as follows: 8 mm slice-thickness, 3.5-3.9 ms repetition time, 14 to 16 K-space lines per segment, 30 phases per cardiac cycle with view sharing, field-of-view (FOV) ranging from 32 to 38 cm with a phase FOV of 0.9, and a 224×224 matrix. LV end-diastolic volume, LV end-systolic volume, stroke volume, LV ejection fraction and LVM were determined on the contiguous SSFP short-axis slices, using dedicated software (MASS™, Medis, The Netherlands). LVM was determined at end-diastole, and papillary muscles and trabeculations were excluded for LVM and volume measurements (Codreanu et al., 2007; Mandry et al., 2009). CRI, indicating concentric LV remodeling, is represented by the ratio of LVM/LV end-diastolic volume (Bluemke et al., 2008; Cheng et al., 2008). Alfakih et al. defined LVH, assessed by MRI, as: women: >60 g/m² and men >77 g/m² (Alfakih et al., 2003).

Determination of Fat-Free Mass (Dual-Energy X-Ray Absorptiometry (DEXA))

Body composition was estimated from the attenuation of X-rays pulsed synchronously between 40 and 100 keV using a LUNARS DPX-IQ system (LUNARS Corporation, Madison, Wis., USA) (He et al., 1999). Total fat mass, total lean mass and total bone mass were expressed in kg. Percentage of fat mass and fat free mass were evaluated using the proportions of total fat mass. Fat-free mass is an interesting criterion index in obese patients since it removes body fat. This index has never been used in studies of LVM by MRI but was in TTE (Quilliot et al., 2005; Bella et al., 1998).

Vascular Phenotyping

Three consecutive BP measurements were performed first during the screening visit then before the echotracking and MRI (about one month apart), and the mean of the two last readings was recorded each time. Therefore, 4 measurements were taken into account in this analysis. All were performed after an extended rest period of at least 30 minutes. Carotid intima-media thickness and external pulse wave velocity were assessed as we previously reported (Kearney-Schwartz et al., 2009).

Biological Phenotyping

Blood samples were taken off between 8 and 10 am after maintaining the supine position for 30 minutes: fasting glucose, oral glucose tolerance test (to exclude diabetic patients), and glycated haemoglobin serum creatinine [estimated glomerular filtration rate by the MDRD formula (Levey et al., 1999)], serum protein, lipoprotein A, ultra-sensitive C-reactive protein, lipid profile (triglycerides, total cholesterol, high density lipoprotein (HDL) and low density lipoprotein (LDL)) were measured. Samples were stored at −80° C. until assayed. Radioimmunoassay kits (Orion Diagnostica, Espoo, Finland) were used for determinations of serum collagen peptides concentrations (PINP: aminoterminal propeptide of type I procollagen, PIIINP: aminoterminal propeptide of type III procollagen, biomarkers of collagen synthesis; ICTP: type 1 collagen telopeptide, biomarker of collagen degradation) as we reported (Iraqi et al., 2009; Zannad et al., 2010). They were performed by technicians blind for patients group.

Statistical Analysis

All tests were conducted using SAS software R9.1.3 (SAS Institute, Cary, N.C., USA). The bilateral significance level was set at 5%. The sample size allowed to detect with a 80% power a difference of 0.45 SD between groups.

Comparisons were carried out using the nonparametric Mann-Whitney.

Multivariate linear regressions were conducted on LVM (g, g/m² g/kg), CRI, pulse wave velocity, intima-media thickness, E′, E′ <10 cm/s and E/E′. Significant covariates were selected using an interactive backward stepwise method. Intercorrelated variables (e.g. SBP, DBP and mean BP) were tested separately in the models. Each biomarker was tested individually in separate models. The conditions of validity of the models (linearity, normality of residuals, homoscedasticity, absence of interaction and collinearity, impact of outliers) were thoroughly checked for each model. The factors associated with DD were identified using logistic regression. When the assumption of linearity of the association between DD and continuous variable could not be accepted, the factor was dichotomized according to the median.

The results are presented as mean±standard deviation (m±SD), regression coefficient or odds ratio (confidence interval 95%).

Results

Study population features

192 subjects were recruited. Twenty-two (11 AO, 11 healthy volunteers) were excluded because of the finding of arterial hypertension during the initial visit (2 AO, 10 “healthy volunteers”), antihypertensive therapy (3 AO using beta-blockers and 1 healthy volunteers an α-blocker), thyroid hormone medication (6 AO), and one patient lost to follow-up between the two visits. Therefore, our final study population included 169 subjects: 116 subjects in the AO group and 53 healthy volunteers.

The clinical, biological characteristics of both groups are presented in Table 1.

As expected from the inclusion criteria there was no significant difference regarding age and the gender ratio, whereas a significant increase in body mass index (p<0.0001), waist circumference (p<0.0001), and the waist/hip ratio (p<0.0001) were found in the AO population. On average, study participants did not exhibit BP in the hypertensive range, but SBP (p<0.0001) and DBP (p=0.0002) were significantly higher in the AO group, with also a higher heart rate (p<0.0001).

A significant increase of PINP (p<0.0001), accompanied by a decrease of ICTP (p<0.0001) concentrations were observed in AO.

The structural and functional cardiovascular data are summarized in Table 1. AO displayed a ventricular remodeling, as assessed by a significant increase in LVM (p=0.003), without reaching a LVH. Moreover, a CRI increase (p=0.004) secondary to the increase in LVM [no significant difference in LV end diastolic volume (p=0.90)] was observed in the AO group. The LV ejection fraction was preserved in both groups (p=0.98). E′ was significantly lower by about 30% in the AO group (p<0.0001). Forty-six per cent of AO patients presented DD (grade I) compared to only 4% of controls. Two percent of AO group were already classified grade II, while 23% were in an intermediate group between groups I and II. Subjects with DD displayed a higher LVM (p=0.003) and a higher DBP (p<0.0001), while the latter remaining within the normal range (data not shown). Furthermore LV filling pressures (evaluated by E/E′ ratio) were significantly higher in obese patients (p=0.0003), but still in the normal range.

The two groups did not significantly differ in terms of vascular parameters [i.e. PWV (p=0.96), despite a trend toward increased intima-media thickness in the AO group (p=0.097)]. Only 5% and 1% of the total population respectively presented an elevation of the intima-media thickness (>0.90 mm) or of the PWV (>12 m/s) (Mancia et al., 2007).

Association of PIIINP with Diastolic Dysfunction

Collagen peptides were not associated with LVM or CRI. PIIINP concentration was positively associated with DD (p=0.038) independently from the DBP (>74 mmHg, p<0.001) (Table 2).

Discussion:

The present study provides a number of novel findings. AO subjects exhibited an increased LVM, without LVH, as well as a more pronounced concentric CR.

PIIINP serum concentrations, reflecting myocardial fibrosis was found to be associated with DD. These findings were all observed after adjustment for conventional risk factors (metabolic syndrome components) and systemic inflammation, which were significantly higher in AO. Finally, we did not observe any modification in vascular structure and function in obese subjects.

Extra-Cellular Matrix Turnover

The inventors have investigated whether the increase in LVM and CR was correlated with markers of myocardial collagen fibrosis, the goal being to pinpoint the factors underlying the occurrence of DD in this population prone to HF. To our knowledge, our study is the first one to show that a collagen peptide, PIIINP, a marker of collagen synthesis, is a positive explanatory factor of DD in asymptomatic patients without LVH, even though concentrations did not differ significantly between the two groups. Of note PINP was found increased and ICTP decreased in AO, thereby suggesting an ongoing cardiovascular profibrotic process. Mak et al. reported a progressive increase in markers of collagen turnover in preserved systolic function HF patients, which was prevented by eplerenone (Mak et al., 2009). Martos et al. showed that in hypertensive patients, ICTP, PINP, and PIIINP concentrations were greater in patients with diastolic HF (Martos et al., 2007).

In this study, collagen peptides were not correlated with LVM or CR. The transformation of the extracellular matrix toward a more substantial collagen component induces a loss of relaxation potentiated by the increase in LVM, and possibly contributing to the development of LV DD.

Diastolic Dysfunction and Diastolic Blood Pressure

Finally, we were also able to identify an additional factor associated with the occurrence of DD: a DBP ≧74 mmHg in non-hypertensive patients. In our study population, subjects with DD had a higher LVM, without LVH, as well as a higher DBP whilst remaining within normal limits. While it has already been repeatedly demonstrated that hypertension via LVH leads to DD, this is first instance, to our knowledge, to show that a normal DBP in non-hypertensive patients on average, is shown as a determinant of the existence of DD. SBP and DBP were also independent predictors of LVM and CRI respectively, independent from the study groups.

TABLE 1
Study population features

| | Abdominal obesity | | | | |
|---|---|---|---|---|---|
| | group | | Control group | | |
| | n | Mean ± DS | n | Mean ± DS | p |
| Clinical characteristics | | | | | |
| Sex M/F | 116 | 50/50 | 53 | 45/55% | 0.57 |
| Age (years) | 116 | 55 ± 6 | 53 | 54 ± 6 | 0.18 |
| BMI (kg/m²) | 116 | 31.7 ± 3.4 | 53 | 22.4 ± 2.0 | <0.0001 |
| Body surface area (m²) | 116 | 2.07 ± 0.18 | 53 | 1.73 ± 0.14 | <0.0001 |
| Waist size (cm) | 116 | 103 ± 10 | 53 | 78 ± 8 | <0.0001 |
| Waist/hip ratio | 116 | 0.94 ± 0.10 | 53 | 0.82 ± 0.09 | <0.0001 |
| Mean SBP (mmHg) | 116 | 128 ± 16 | 53 | 116 ± 11 | <0.0001 |
| Mean DBP (mmHg) | 116 | 77 ± 11 | 53 | 71 ± 6 | 0.0002 |
| Mean MBP (mmHg) | 116 | 94 ± 12 | 53 | 86 ± 7 | <0.0001 |
| Mean HR (bpm) | 116 | 70 ± 10 | 53 | 62 ± 8 | <0.0001 |
| Biological | | | | | |
| characteristics | | | | | |
| Triglycerides (mmol/l) | 116 | 1.62 ± 1.16 | 53 | 0.88 ± 0.43 | <0.0001 |
| Total cholesterol | 116 | 5.78 ± 0.99 | 53 | 5.44 ± 0.83 | 0.045 |
| (mmol/l) | | | | | |
| HDL Cholesterol | 116 | 1.42 ± 0.38 | 53 | 1.60 ± 0.36 | 0.002 |
| (mmol/l) | | | | | |
| LDL Cholesterol | 116 | 3.63 ± 0.88 | 53 | 3.45 ± 0.73 | <0.0001 |
| (mmol/l) | | | | | |
| Fasting glucose | 112 | 5.18 ± 0.95 | 49 | 4.43 ± 1.41 | 0.015 |
| (mmol/l) | | | | | |
| Glycated haemoglobin | 113 | 5.8 ± 0.5 | 50 | 5.6 ± 0.3 | 0.020 |
| (%) | | | | | |
| CRP (mg/l) | 115 | 3.7 ± 5.5 | 52 | 1.2 ± 1.3 | <0.0001 |
| Protidemia (g/l) | 116 | 72 ± 4 | 52 | 69 ± 4 | <0.0001 |
| eGFR (MDRD, ml/ | 116 | 76 ± 10 | 53 | 77 ± 12 | 0.92 |
| min/1.73 m²) | | | | | |
| PINP (ηg/ml) | 115 | 36 ± 16 | 53 | 22 ± 14 | <0.0001 |
| PIIINP (ηg/ml) | 110 | 2.6 ± 1.3 | 53 | 3.4 ± 6.7 | 0.082 |
| ICTP (ηg/ml) | 115 | 3.9 ± 1.0 | 53 | 4.6 ± 0.9 | <0.0001 |
| Cardiac phenotyping | | | | | |
| LVM (g)* | 93 | 97 ± 25 | 47 | 84 ± 21 | 0.003 |
| LVMi (g/m²)* | 93 | 47 ± 10 | 47 | 48 ± 10 | 0.21 |
| LVM FFM (DEXA) | 93 | 1.79 ± 0.28 | 47 | 1.78 ± 0.28 | 0.86 |
| (g/kg)* | | | | | |
| LVEF (%)* | 93 | 60 ± 6 | 47 | 59 ± 7 | 0.98 |
| LVEDV (ml)* | 93 | 142 ± 29 | 47 | 141 ± 25 | 0.90 |
| LVESV (ml)* | 93 | 58 ± 19 | 47 | 57 ± 15 | 0.93 |
| LVEV (ml)* | 93 | 84 ± 15 | 47 | 84 ± 17 | 0.83 |
| CRI = LVM/LVEDV | 93 | 0.69 ± 0.16 | 47 | 0.60 ± 0.10 | 0.004 |
| (g/ml)* | | | | | |
| E (cm/s)^{†} | 116 | 66 ± 16 | 52 | 74 ± 15 | 0.005 |
| A (cm/s)^{†} | 116 | 60 ± 15 | 52 | 53 ± 10 | 0.002 |
| E/A^{†} | 116 | 1.13 ± 0.31 | 52 | 1.41 ± 0.31 | <0.0001 |
| E′ (cm/s)^{†} | 112 | 10.4 ± 2.5 | 52 | 13.7 ± 2.5 | <0.0001 |
| A′ (cm/s)^{†} | 113 | 11.0 ± 2.6 | 52 | 9.4 ± 2.6 | 0.0002 |
| E/E′^{†} | 113 | 6.6 ± 1.7 | 52 | 5.5 ± 1.5 | 0.0003 |
| Vascular phenotyping | | | | | |
| PWV (m/s) | 95 | 7.9 ± 1.7 | 48 | 8.0 ± 1.4 | 0.96 |
| IMT (mm) | 97 | 0.66 ± 0.15 | 48 | 0.61 ± 0.12 | 0.097 |

BMI: body mass index,
CRP: C reactive protein,
CRI = cardiac remodeling index,
DBP: diastolic blood pressure,
eGFR: estimated glomerular filtration rate,
F: female,
HDL: High density lipoprotein,
HR: heart rate,
bpm: beats per minute,
ICTP: type 1 collagen telopeptide,
IMT: intima-media thickness,
LDL: Low density lipoprotein,
LVEDV: LV end-diastolic volume,
LVEF: left ventricular ejection fraction,
LVESV: LV end-systolic volume,
LVEV: LV ejection volume,
LVM: left ventricular mass,
M: male,
LVM FFM: LVM indexed by fat free mass,
LVMi: LVM indexed by BSA (Boyd),
MBP: mean blood pressure,
MDRD: Modification in Diet Renal Disease,
PINP: aminoterminal propeptide of type I procollagen,
PIIINP aminoterminal propeptide of type III procollagen,
PWV: pulse wave velocity,
SBP: systolic blood pressure,
*evaluated by MRI
^{†}evaluated by TTE

TABLE 2
Factors associated with diastolic dysfunction in multivariate analysis.

| Diastolic dysfunction [E′ (<10 cm/s)]; | | | |
|---|---|---|---|
| n = 50/158 | OR (95% CI) | | p |
| Abdominal obesity | 11.41 | (2.43-53.59) | 0.002 |
| PIIINP ≧2.6 ng/ml | 2.44 | (1.05-5.69) | 0.038 |
| Triglycerides (>1.11 mmol/l) | 2.77 | (1.14-6.76) | 0.025 |
| DBP (≧74 mmHg) | 5.55 | (2.35-13.14) | <0.0001 |

DBP: diastolic blood pressure,
PIIINP aminoterminal propeptide of type III procollagen,
* OR (95% CI): odds ratio (95% confidence interval);
p: p-value from logistic regression

### REFERENCES

Throughout this application, various references describe the state of the art to which this invention pertains. The disclosures of these references are hereby incorporated by reference into the present disclosure.

Holt R I G. International Diabetes Federation re-defines the metabolic syndrome. Diabetes Obes Metab 2005; 7(5):618-620.

Bluemke D A, Kronmal R A, Lima J A C, et al. The relationship of left ventricular mass and geometry to incident cardiovascular events: the MESA (Multi-Ethnic Study of Atherosclerosis) study. J. Am. Coll. Cardiol 2008; 52(25):2148-2155.

Cheng S, Fernandes V R S, Bluemke D A, McClelland R L, Kronmal R A, Lima J A C. Age-related left ventricular remodeling and associated risk for cardiovascular outcomes: the Multi-Ethnic Study of Atherosclerosis. Circ Cardiovasc Imaging 2009; 2(3):191-198.

Zannad F, Alla F, Dousset B, Perez A, Pitt B. Limitation of excessive extracellular matrix turnover may contribute to survival benefit of spironolactone therapy in patients with congestive heart failure: insights from the randomized aldactone evaluation study (RALES). Rales Investigators. Circulation 2000; 102(22):2700-2706.

Iraqi W, Rossignol P, Angioi M, et al. Extracellular cardiac matrix biomarkers in patients with acute myocardial infarction complicated by left ventricular dysfunction and heart failure: insights from the Eplerenone Post-Acute Myocardial Infarction Heart Failure Efficacy and Survival Study (EPHESUS) study. Circulation 2009; 119(18):2471-2479.

Codreanu A, Djaballah W, Angioi M, et al. Detection of myocarditis by contrast-enhanced MRI in patients presenting with acute coronary syndrome but no coronary stenosis. J Magn Reson Imaging 2007; 25(5):957-964.

Mandry D, Lapicque F, Odille F, et al. Multicompartmental analysis of late contrast enhancement in areas of myocardial infarction supplied by chronically occluded coronary arteries. J Magn Reson Imaging 2009; 29(1):78-85.

Alfakih K, Plein S, Bloomer T, Jones T, Ridgway J, Sivananthan M. Comparison of right ventricular volume measurements between axial and short axis orientation using steady-state free precession magnetic resonance imaging. J Magn Reson Imaging 2003; 18(1):25-32.

He M, Li E T, Kung A W. Dual-energy X-ray absorptiometry for body composition estimation in Chinese women. Eur J Clin Nutr 1999; 53(12):933-937.

Quilliot D, Alla F, Böhme P, et al. Myocardial collagen turnover in normotensive obese patients: relation to insulin resistance. Int J Obes (Lond) 2005; 29(11):1321-1328.

Bella J N, Devereux R B, Roman M J, et al. Relations of left ventricular mass to fat-free and adipose body mass: the strong heart study. The Strong Heart Study Investigators. Circulation 1998; 98(23):2538-2544.

Kearney-Schwartz A, Rossignol P, Bracard S, et al. Vascular structure and function is correlated to cognitive performance and white matter hyperintensities in older hypertensive patients with subjective memory complaints. Stroke 2009; 40(4):1229-1236.

Levey A S, Bosch J P, Lewis J B, Greene T, Rogers N, Roth D. A more accurate method to estimate glomerular filtration rate from serum creatinine: a new prediction equation. Modification of Diet in Renal Disease Study Group. Ann. Intern. Med 1999; 130(6):461-470.

Zannad F, Rossignol P, Iraqi W. Extracellular matrix fibrotic markers in heart failure. Heart Fail Rev 2010; 15(4):319-329.

Mancia G, De Backer G, Dominiczak A, et al. 2007 Guidelines for the management of arterial hypertension: The Task Force for the Management of Arterial Hypertension of the European Society of Hypertension (ESH) and of the European Society of Cardiology (ESC). Eur. Heart J 2007; 28(12):1462-1536.

Mak G J, Ledwidge M T, Watson C J, et al. Natural history of markers of collagen turnover in patients with early diastolic dysfunction and impact of eplerenone. J. Am. Coll. Cardiol 2009; 54(18):1674-1682.

Martos R, Baugh J, Ledwidge M, et al. Diastolic heart failure: evidence of increased myocardial collagen turnover linked to diastolic dysfunction. Circulation 2007; 115(7):888-895.

Zile M R. Heart failure with preserved ejection fraction: is this diastolic heart failure? J Am Coll Cardiol. 2003; 7; 41(9):1519-22.

Köhler G, Milstein C. Continuous cultures of fused cells secreting antibody of predefined specificity. Nature. 1975 Aug. 7; 256(5517):495-7.

1. A method for detecting diastolic dysfunction in a subject without clinical signs of heart failure, comprising measuring a PIIINP level in a blood sample obtained from said subject by
contacting the blood sample with a labeled binding partner capable of selectively interacting with PIIINP present in the blood sample, thereby forming a binding partner-PIIINP complex, and
detecting the binding partner-PIIINP complex.

2. The method according to claim 1 further comprising
comparing the PIIINP level in the blood sample with a reference value, wherein detecting a differential in the PIIINP level between the blood sample and the reference value is indicative of detecting diastolic dysfunction in a subject without clinical signs of heart failure.

3. A method for preventing or treating diastolic dysfunction in a subject without clinical signs of heart failure in need thereof, comprising the steps of:
i) detecting diastolic dysfunction in said subject by measuring a PIIINP level in a blood sample obtained from said subject
ii) comparing the PIIINP level in the blood sample with a reference value, wherein detecting a differential in the PIIINP level between the blood sample and the reference value is indicative of detecting diastolic dysfunction in a subject without clinical signs of heart failure, and, if a differential is detected then,
iii) administering to said subject a therapeutically effective amount of an inhibitor of the renin angiotensin aldosterone system.

4. The method of claim 3, wherein measuring the PIIINP level is performed by
contacting the blood sample with a labeled binding partner capable of selectively interacting with PIIINP present in the blood sample, thereby forming a binding partner-PIIINP complex, and
detecting the binding partner-PIIINP complex.
